(19) United States
(12) Patent Application Publication
(10) Pub. No.: US 2014/0287997 A1
(43) Pub. Date: Sep. 25, 2014

(54) USE OF GROWTH HORMONE OR GROWTH HORMONE RECEPTOR AGONISTS TO PREVENT OR TREAT STRESS-SENSITIVE PSYCHIATRIC ILLNESS

(71) Applicant: Massachusetts Institute of Technology, Cambridge, MA (US)

(72) Inventor: Ki Ann Goosens, Cambridge, MA (US)

(73) Assignee: Massachusetts Institute of Technology, Cambridge, MA (US)

(21) Appl. No.: 14/212,365

(22) Filed: Mar. 14, 2014

Related U.S. Application Data
(60) Provisional application No. 61/789,053, filed on Mar. 15, 2013.

(51) Int. Cl. A61K 38/27; A61K 31/7088

(52) U.S. Cl. CPC A61K 38/27; A61K 31/7088
USPC 514/11.3; 514/44 R

(57) ABSTRACT

The invention relates to methods for treating stress sensitive condition, particularly depression. For instance a subject having or at risk of having a stress-sensitive condition such as depression may be treated with a growth hormone (GH) or an agonist thereof in an effective amount to treat depression.

### RELATED APPLICATIONS

This application claims priority under 35 U.S.C. §119(e) to U.S. Provisional Application Ser. No. 61/789,053, entitled “USE OF AGONISTS OF GROWTH HORMONE OR GROWTH HORMONE RECEPTOR TO PREVENT OR TREAT STRESS-SENSITIVE PSYCHIATRIC ILLNESS” filed on Mar. 15, 2013, which is herein incorporated by reference in its entirety.

### FEDERALLY SPONSORED RESEARCH

This invention was made with government support under Grant No. MH084966 awarded by the National Institutes of Health and under Contract No. W911NF-10-1-0059 awarded by the U.S. Army. The government has certain rights in the invention.

### BACKGROUND OF INVENTION

Stress is defined by a constellation of responses that occur when the body's ability to cope with a series of demands is exceeded (1). Stress exposure can vary in duration, and it is clear that stress “load”, defined by both the length of exposure as well as the number of stressors present, plays a role in determining the consequences of stress (2). Short-term stress is thought to recruit adaptive responses that promote coping and resilience. However, the mechanisms for driving adaptive change may be difficult to maintain in the face of repeated challenge, and maladaptations can occur when stress is prolonged (3). For example, high stress load is a risk factor for the development of numerous types of affective mental illness, particularly those involving fear and anxiety (4-6). Despite an abundant literature on the effects of stress in the brain, most studies have focused on the effects of acute stress. Thus, the mechanisms that lead to maladaptations following chronic stress exposure are unclear.

While there are many brain regions that are altered by stress and mediate stress-related changes in behavior, the hippocampus is the region in which the effects of stress are best characterized. The hippocampus plays a role in many types of memory (7), and is also linked to affective regulation (8, 9). Acute stress can both enhance and impair hippocampal function. For example, acute stress can increase (10) or decrease (11) hippocampal dendritic spine density. Acute stress can also enhance (12) or impair (13) hippocampus-dependent cognition, perhaps depending on the level of arousal attained during the stress (14). In contrast, chronic stress generally produces dendritic retraction in hippocampus (15-18), and impairs performance on hippocampus-dependent memory tasks (19-21). These changes are thought to be mediated, in part, by stress hormone-induced downregulation of growth factors, such as brain-derived neurotrophic factor, in neurons (22).

### SUMMARY OF INVENTION

In some aspects the invention is a method for treating depression in a subject, by administering to a subject having depression a growth hormone or an agonist thereof in an effective amount to treat depression. In some embodiments the subject is administered growth hormone. In other embodiments the subject is administered a growth hormone agonist. The growth hormone agonist may be, for instance, a nucleic acid vector, wherein the nucleic acid vector encodes growth hormone or functional analogs thereof.

In some embodiments the growth hormone or agonist thereof is delivered to the hippocampus.

In other embodiments the depression is a depressive disorder, a major depressive disorder, complicated grief, dysthymia, post-partum depression, or psychotic depression. The depression may be associated with chronic stress. In some embodiments the growth hormone or agonist thereof is administered while the subject is experiencing the stress.

In other embodiments the growth hormone or agonist thereof is administered only during the time that the subject is experiencing the stress. In some embodiments the growth hormone or agonist thereof is administered before, during and/or after exposure of the subject to chronic stress.

The growth hormone or agonist thereof may be administered to the subject in a sustained release device.

In some embodiments the subject is diagnosed as having depression but not other stress sensitive disorders.

Optionally, the growth hormone or agonist thereof is administered systemically such as intravenously. In other embodiments the growth hormone or agonist thereof is administered to the subject orally. Use of a compound of the invention for treating a stress sensitive condition or for treating depression is also provided as an aspect of the invention.

In some embodiments the growth hormone or agonist thereof, is growth hormone, human growth hormone, recombinant growth hormone, small molecule GH agonist, or a peptide GH agonist. The peptide GH agonist in some embodiments is a growth hormone fusion protein or antibody to GH, a pegylated GH, or a GH receptor agonist monomers. In other embodiments the GH agonist is a sustained-release formulation of GH. In some embodiments an extracellular domain of the GH receptor (GHBP) is coadministered with the GH or agonist thereof.

A method for manufacturing a medicament of a compound of the invention for treating depression is also provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. For purposes of clarity, not every component may be labeled in every drawing. In the drawings:

FIG. 1 shows that chronic stress reduces hippocampal growth hormone (GH). Hippocampal GH levels were assayed seven days after a two week period of immobilization stress (STR) or handling (NS). GH was significantly lower in the hippocampi of stressed rats relative to unstressed rats.

FIG. 2 shows the construction of an HSV-1 viral vector to overexpress GH. a) The full-length gene for presomatotropin was cloned into an HSV-1 amplicon under the control of the HSV α-4 promoter. eGFP was co-expressed via the HSV α-22 promoter. b) GH protein expression was confirmed in vitro. Vero cells were infected with GH virus at increasing MOIs. As the MOI increased, progressively higher levels of both GH and eGFP were detected. c) The viral vector was infused into the dorsal hippocampus of rats. A representative infection, showing high levels of expression in pyramidal cells of CA1, is shown.

FIG. 3 shows overexpression of hippocampal GH rescues stress-related impairments in auditory trace conditioning. Two weeks of daily immobilization stress (STR) or handling (NS) were administered to rats. Twenty-four hours after the last session, GH or GFP virus was infused bilaterally into the dorsal hippocampus. a) After three days of recovery, auditory trace conditioning was administered. Stress impaired trace conditioning, and this effect was reversed by intra-hippocampal GH. b) Long-term contextual fear memory was measured the next day by returning rats to the conditioning context for 5 m. Stress impaired contextual fear memory, and intra-hippocampal GH expression partially reversed this impairment. c) Long-term auditory fear memory was measured the following day by placing the rats in a novel context and presenting four tones in the absence of footshock. Stress impaired auditory fear memory, and intra-hippocampal GH expression fully reversed this effect. * indicates p<0.05 in a post-hoc comparison.

FIG. 4 shows that overexpression of hippocampal GH rescues stress-related impairments in contextual conditioning. Ten days of daily immobilization stress (STR) or handling (NS) were administered to rats. Twenty-four hours after the last session, GH or GFP virus was infused bilaterally into the dorsal hippocampus. a) After three days of recovery, contextual fear conditioning was administered. Stress slowed contextual fear acquisition, and this was prevented by intra-hippocampal GH. In contrast, intra-hippocampal GH had no effect in unstressed control rats. b) The next day, the rats were returned to the context for an 8 m context extinction session. Stress impaired long-term contextual fear memory, and this impairment was rescued by expression of GH in the dorsal hippocampus. In contrast, intra-hippocampal GH produced a mild impairment of long-term contextual fear memory in unstressed control rats. * indicates p<0.1 in a post-hoc comparison.

FIG. 5 shows that amygdalar growth hormone is increased by chronic stress, is sufficient to enhance fear memory and is necessary for the fear potentiating effects of ghrelin receptor stimulation. a) Rats received either daily handling (no stress (NS)) or immobilization stress (STR) for 14 days Animals were killed 24 h after the last stress or handling session and the basolateral complex of the amygdala (BLA) was dissected. Growth hormone (GH) levels in the BLA were measured using enzyme-linked immunosorbent assay (ELISA). b) The herpes simplex virus (HSV)-based viral vectors expressing either green fluorescent protein (GFP; CON) or recombinant rat GH (rGH) was infused in the BLA and expression was assessed after behavioral testing was complete. Representative GFP expression from two rats is shown. c) Auditory Pavlovian fear conditioning was performed 3 days after HSV virus infusions. d) Long-term fear memory was assessed by placing the animals in a novel context and measuring conditional freezing following tone presentation 48 h after the conditioning session. e) Short-term BLA cell cultures were used to measure GH release following treatment with a ghrelin receptor agonist (MK) or vehicle (VEH). f) Adeno-associated virus (AAV) constructs were generated to examine the contribution of GH-mediated signaling in the BLA to ghrelin-induced potentiation of fear. g) Following infusion of the AAVs into the BLA and recovery, rats received 10 days of systemic injection of either a ghrelin receptor agonist (MK) or vehicle (VEH). After 24 h, auditory Pavlovian fear conditioning was administered to all rats. h) Long-term fear memory was assessed by placing the animals in a novel context and measuring conditional freezing following tone presentation 48 h after the conditioning session. Scale bar, is 500 mm. All data are mean±s.e.m. *P<0.05, **P<0.01 in planned comparisons.

FIG. 6 shows that increasing GH levels in the hippocampus leads to increased spine density. a and b) Images of increased spine density in the hippocampus. c) A graph showing that GH overexpression doubles dendritic spine density in hippocampus.

FIG. 7 shows a bar graph assessing sucrose preference (measuring hedonia) as a measure of depression using vehicle, low dose MK0677 or high dose MK0677.

FIG. 8 shows a bar graph assessing forced swim as a measure of depression using vehicle, low dose MK0677 or high dose MK0677.

### DETAILED DESCRIPTION

Growth hormone (GH) is a hormone released into the circulating blood stream by the pituitary, but it is also synthesized by the hippocampus and other brain regions (23, 24). Within the hippocampus, application of exogenous GH is sufficient to induce synaptic plasticity (25). Exogenous GH also facilitates hippocampal synaptic transmission (26, 27) and hippocampus-dependent eyeblink conditioning is associated with enhanced GH protein synthesis in hippocampal cells (28). Interestingly, hippocampal GH levels are stress-sensitive: GH gene transcription is regulated by glucocorticoid stress hormones, and GH protein levels are increased one day after an acute stress exposure (30). However, these studies are largely correlational. It was discovered according to the invention that higher levels of hippocampal GH may promote hippocampal function. The examples provided herein have examined hippocampal GH following chronic stress. The relationship between GH and stress-related changes in hippocampal function was explored by using viral-mediated gene transfer to manipulate GH levels in stressed and unstressed rats prior to training on one of two hippocampus-dependent behavioral tasks.

Although growth hormone (GH) is synthesized in hippocampal neurons, where it is regulated by stress exposure, its function is poorly characterized. The invention is based at least in part on the finding that a regimen of chronic stress that impairs hippocampal function in rats also leads to a profound decrease in hippocampal GH levels. Restoration of hippocampal GH in the dorsal hippocampus completely reversed stress-related impairments in both auditory trace fear conditioning and contextual fear conditioning, without affecting hippocampal function in unstressed control rats. GH overexpression reversed stress-induced decrements in both fear acquisition and long-term fear memory. These results show that loss of hippocampal GH contributes to hippocampal dysfunction following prolonged stress, and demonstrate that restoring hippocampal GH levels following stress can promote stress resilience. It is also demonstrated herein that increasing GH levels in the hippocampus leads to increased spine density. Since stress and depression both cause hippocampal atrophy, this data suggest that GH or agonists thereof are useful in treating stress and depression. Low and high doses of the ghrelin receptor agonist also were demonstrated to lead to anhedonia, as a measure of depression. Repeated ghrelin receptor stimulation (mimicking one effect of chronic stress, which is to enhance the circulating levels of ghrelin and thus ghrelin signaling) induces depressive behaviors in rats.

Thus in some aspects the invention is a method for treating depression in a subject by administering to the subject having depression a growth hormone or an agonist thereof in an effective amount to treat depression.

Growth hormone or agonist thereof, as used herein, refers to a compound that activates the growth hormone receptor to induce receptor signaling. These compounds include growth hormone, human growth hormone, recombinant growth hormone, small molecule and peptide agonist thereof, including growth hormone fusion proteins such as those described in U.S. Pat. No. 8,293,709 and antibodies to GH such as those described in U.S. Pat. No. 4,857,637, and nucleic acids expressing or encoding growth hormone or functional analogs thereof. Human growth hormone (hGH) is a 22,000 Dalton pituitary hormone known to exhibit a multitude of biological effects, including linear growth (somatogenesis), lactation, activation of macrophages, and insulin-like and diabetogenic effects, among others. Chawla, Annu. Rev. Med., 34:519 (1983); Edwards et al, Science, 239:769 (1988); Isaksson et al, Annu. Rev. Physiol., 47:483 (1985); Thomer et al, J. Clin. Invest., 82:745 (1988); Hughes et al, Annu. Rev. Physiol., 47:469 (1985). These biological effects derive from the interaction between hGH and specific cellular receptors. Growth hormone agonists include compounds such as those described in U.S. Pat. No. 7,632,809. Long-acting GH agonist preparations, include pegylated GH (Clark, R. et al. Long-acting growth hormones produced by conjugation with polyethylene glycol., Journal of Biological Chemistry. 271, 21969-21977 (1996).) and sustained-release formulations. (Cook, D. M. et al. The pharmacokinetic and pharmacodynamic characteristics of a long-acting growth hormone (GH) preparation (nutropin depot) in GH-deficient adults, J Clin Endocrinol Metab. 87, 4508-14 (2002), Reiter, E. O. et al. A multicenter study of the efficacy and safety of sustained release GH in the treatment of naive pediatric patients with GH deficiency, J Clin Endocrinol Metab. 86, 4700-6 (2001) and Jostel, A. et al., A new sustained-release preparation of human growth hormone and its pharmacokinetic, pharmacodynamic and safety profile. Clin Endocrinol (Oxf). 62, 623-7 (2005).). Growth hormone receptor agonist monomers are described in US Patent Application 2004/0033948. Growth hormone receptor modulators, including GH agonists are also described in Birzniece V. et al., Rev Endocr Metab Disord. 2009 June; 10(2):145-56. Each of the above-identified publications, patents and patent applications is incorporated by reference herein for the teachings of GH agonists.

The extracellular domain of the GH receptor (GHR) is proteolytically cleaved and circulates as a binding protein (GHBP). Under physiological conditions GH is in part bound in the circulation in a 1:1 molar ratio by GHBP and this complex appears to be biologically inactive, protected from clearance and degradation. Co-administration of separately purified GHBP with GH in a 1:1 ratio can augment the anabolic actions of GH. Thus, co-administration of GHBP is envisioned as part of the invention.

The growth hormone agonist could either be a compound that acts directly on the GH receptor, or a compound that acts indirectly. A compound that acts indirectly is one that boosts signaling cascades downstream of the GH receptor, or a compound that enhances the release of endogenous neuronal growth hormone or stabilizes or enhances the expression or activity of endogenous GH. A compound that enhances expression of endogenous GH includes for instance GH gene activators as well as inhibitory molecules that act on compounds that inhibit or downregulate the production or expression of GH.

The growth hormone or agonist thereof may be administered to the subject systemically or locally. For instance it may be delivered to or targeted to the hippocampus.

The growth hormone or agonist thereof is useful for preventing the development of the depression, and for treating depression. As such it can be administered to the subject either prior to or during the course of the depression, or following a stress exposure that leads to depression. An inhibitory nucleic acid may be used, for instance, to down regulate the expression of a molecule that inhibits GH. The inhibitory nucleic acid may be, for instance, an siRNA or an antisense molecule that inhibits expression of a GH protein expression inhibitor. The inhibitory nucleic acids may be designed using routine methods in the art.

Depression has various forms which are defined and potentially separately diagnosed according to criteria given in handbooks for psychiatry, for example in the Diagnostic and Statistical Manual of Mental Disorders 4th edition (DSM-IV) published by the American Psychiatric Association, Washington, D.C. (1994). In the DSM-IV, depressive disorders are classified under mood disorders and are divided into three types: major depressive disorder, dysthymic disorder and depressive disorder not otherwise specified (or “atypical”). In general, regardless of whether or not the depressive syndrome is melancholic, atypical, or some admixture of the two, a diagnosis of major depression is given when depressed mood is present, or loss of interest or pleasure in all activities is present, for at least two weeks.

Clinical depression is common, occurring in about one in five people during their lifetime and among the top four most common illnesses internationally as listed by the World Health Organization. Depression may involve persistent sadness, loss of self-esteem, difficulty concentrating, guilt, hopelessness, avoiding other people, loss of appetite, lack of enjoyment, and suicidal thoughts.

Major depressive disorder and dysthymic disorder are differentiated based on chronicity, severity and persistence. If less severe or incapacitating, depressed mood is considered dysthymia. Depressed mood can occur in the form of a cycling mood abnormality such as bipolar mood disorder, cyclothymia, or menstrual-related mood disorder. In dysthymic disorder the depressed mood must be present most days over a period of two years.

Aspects of the invention relate to the effects of stress and, in particular, chronic stress. As used herein, “stress” refers to a physical, chemical or emotional factor or combination of factors that causes bodily or mental tension and that may be a factor in disease causation. It should be appreciated that any form of stress can be compatible with aspects of the invention. Exposure to stress can be chronic or acute. As used here, “chronic stress” refers to a state of prolonged tension from internal or external stressors, which may cause various physical manifestations. The effects of chronic and acute stress can be different. Several non-limiting examples of situations where a subject could be exposed to chronic stress include military service such as a combat mission, and natural disasters, such as participation in a search-and-rescue operation or rebuilding following a natural disaster.

Subjects who are exposed to stress can also develop stress-sensitive disorders. As used herein, a “stress-sensitive disorder” refers to any condition, disease or disorder that results, at least in part, from exposure to stress or is exacerbated, at least in part, from exposure to stress. Non-limiting examples of stress-sensitive disorders treatable according to the instant invention include depression, Depressive Disorder, Major Depressive Disorders, complicated grief, dysthymia, post-partum depression, or psychotic depression. It should be appreciated that other stress-sensitive disorders may be compatible with aspects of the invention.

Aspects of the invention relate to methods by which the effects of recurring stress can be weakened to reduce the potentiating effects of stress on stress-sensitive mental illnesses. Methods associated with the invention comprise administration of a therapeutically effective amount of a GH or agonist thereof to a subject.

The agent can be administered to a subject before, during and/or after exposure to chronic stress. For example, the agent can be administered to a subject in anticipation of exposure to chronic stress, such as prior to participation in a military operation. As such, the agent can protect against the consequences of exposure to chronic stress. The agent can also be administered to a subject during exposure to chronic stress to protect against the consequences of exposure to chronic stress and treat symptoms associated with the effects of chronic stress. The agent can also be administered after exposure to chronic stress to protect against the consequences of exposure to chronic stress and treat symptoms associated with the effects of chronic stress.

Further aspects of the invention relate to determining whether a subject exposed to chronic stress has an increased risk of developing a stress-sensitive disorder. For example, if elevated levels of GH in the amygdala are detected in a subject during or after exposure to chronic stress, the subject may be considered to be at increased risk of developing a depression following exposure to the chronic stress.

Administering a GH or agonist thereof to a subject who will be exposed to chronic stress may reduce the incidence of trauma-induced disorders such as post-traumatic stress disorder (PTSD). Moreover, in the past, most stress-sensitive illnesses have been treated with the same compounds that are used to treat other mental illnesses, such as selective serotonin reuptake inhibitors (SSRIs). However, these drugs do not offer any clinical benefit to a significant number of patients diagnosed with these disorders. Having drugs with a novel mechanism of action, targeting the GHR signaling pathway, may be beneficial for patients who are resistant to traditional avenues of treatment.

The methods of the invention are useful for treating a subject in need thereof. A subject in need thereof can be a subject who will be exposed to chronic stress, is currently exposed to chronic stress or has been exposed to chronic stress. For example, a subject in need thereof may be a subject involved, or who will be involved, in a military operation or combat mission. A subject in need thereof can be a subject having or at risk of depression. For example, a subject can be a patient who is diagnosed with a stress-sensitive disorder, or a subject with a strong familial history of such disorders.

In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms associated with the disorder or the severity of the disease or preventing any further progression of the disease. If the subject in need of treatment is one who is at risk of having a condition, then treating the subject refers to reducing the risk of the subject having the condition or preventing the subject from developing the condition.

A subject shall mean a human or vertebrate animal or mammal including but not limited to a dog, cat, horse, cow, pig, sheep, goat, turkey, chicken, and primate, e.g., monkey.

Therapeutic compounds associated with the invention may be directly administered to the subject or may be administered in conjunction with a delivery device or vehicle. Delivery vehicles or delivery devices for delivering therapeutic compounds to surfaces have been described. The therapeutic compounds of the invention may be administered alone (e.g., in saline or buffer) or using any delivery vehicles known in the art.

The term effective amount of a therapeutic compound of the invention refers to the amount necessary or sufficient to realize a desired biologic effect. For example, an effective amount of a therapeutic compound associated with the invention may be that amount sufficient to ameliorate one or more symptoms of depression in a subject who has been exposed to chronic stress. The effective amount is an amount sufficient to produce therapeutic levels of growth hormone or agonist thereof in the hippocampus. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the invention without necessitating undue experimentation.

Subject doses of the compounds described herein for delivery typically range from about 0.1 μg to 10 mg per administration, which depending on the application could be given daily, weekly, or monthly and any other amount of time there between. The doses for these purposes may range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

In some embodiments a compound of the invention is administered at a dosage of between about 1 and 10 mg/kg of body weight of the mammal. In other embodiments a compound of the invention is administered at a dosage of between about 0.001 and 1 mg/kg of body weight of the mammal. In yet other embodiments a compound of the invention is administered at a dosage of between about 10-100 ng/kg, 100-500 ng/kg, 500 ng/kg-1 mg/kg, or 1-5 mg/kg of body weight of the mammal, or any individual dosage therein.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compound associated with the invention can be administered to a subject by any mode that delivers the therapeutic agent or compound to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, rectal and intracerebroventricular.

For oral administration, the therapeutic compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the invention. The therapeutic agent is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified therapeutic agent may also be prepared in different formulations depending on the type of chemical modification or the type of device employed.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Intra-nasal delivery of a pharmaceutical composition of the present invention is also contemplated. Intra-nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a therapeutic compound of the invention optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agents may be delivered to the brain using a formulation capable of delivering a therapeutic agent across the blood brain barrier. One obstacle to delivering therapeutics to the brain is the physiology and structure of the brain. The blood-brain barrier is made up of specialized capillaries lined with a single layer of endothelial cells. The region between cells are sealed with a tight junction, so the only access to the brain from the blood is through the endothelial cells. The barrier allows only certain substances, such as lipophilic molecules through and keeps other harmful compounds and pathogens out. Thus, lipophilic carriers are useful for delivering non-lipophilic compounds to the brain. For instance, DHA, a fatty acid naturally occurring in the human brain has been found to be useful for delivering drugs covalently attached thereto to the brain (Such as those described in U.S. Pat. No. 6,407,137). U.S. Pat. No. 5,525,727 describes a dihydropyridine pyridinium salt carrier redox system for the specific and sustained delivery of drug species to the brain. U.S. Pat. No. 5,618,803 describes targeted drug delivery with phosphonate derivatives. U.S. Pat. No. 7,119,074 describes amphiphilic prodrugs of a therapeutic compound conjugated to an PEG-oligomer/polymer for delivering the compound across the blood brain bather. Others are known to those of skill in the art.

In some instances it is desirable to deliver the growth hormone or agonist thereof to the subject in the form of a nucleic acid vector. The vector can be targeted to or have targeted expression within the hippocampus for enhancing specific delivery. Thus the treatment methods disclosed herein may involve administering a therapeutically effective amount of a composition that induces the expression of a growth hormone or agonist thereof gene. In some instances, the composition that induces the expression of a growth hormone or agonist thereof gene comprises a vector, such as an isolated plasmid, that expresses the growth hormone or agonist thereof.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes or portions thereof.

An expression vector is one into which a desired sequence may be inserted, e.g., by restriction and ligation such that it is operably joined to regulatory sequences and may be expressed as an RNA transcript. Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays known in the art (e.g., β-galactosidase or alkaline phosphatase), and genes that visibly affect the phenotype of transformed or transfected cells, hosts, colonies or plaques (e.g., green fluorescent protein).

Methods for identifying and obtaining coding sequences for use in the methods disclosed herein are routine in the art. For example, the skilled artisan may search Entrez Gene database using a GeneID or GeneAlias of a growth hormone or agonist thereof. In most cases, links to commercial suppliers (e.g., Open Biosystems) of cDNA's containing the transcripts are provided in the Entrez Gene webinterface, which can be utilized to procure a copy cDNA clone. In other cases, commercial sources (e.g., Sigma Aldrich) can be contacted directly.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Such 5′ non-transcribed regulatory sequences will include a promoter region that includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

In some embodiments, a virus vector for delivering a nucleic acid molecule, an isolated plasmid, is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus to (Davis et al., J. Virol. 70:3781-3787, 1996), Sindbis virus (Pugachev et al., Virology 212:587-594, 1995), lentiviral vectors (Naldini L, et al., Proc Natl Acad Sci USA. 1996 Oct. 15; 93(21):11382-8) and Ty virus-like particle (Allsopp et al., Eur. J. Immunol 26:1951-1959, 1996).

Another virus useful for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other useful viral vectors are based on non-cytopathic eukaryotic viruses in which non-essential genes have been replaced with the gene of interest. Non-cytopathic viruses include certain retroviruses, the life cycle of which involves reverse transcription of genomic viral RNA into DNA with subsequent proviral integration into host cellular DNA. In general, the retroviruses are replication-deficient (i.e., capable of directing synthesis of the desired transcripts, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Clifton, N.J. (1991).

The therapeutic agents of the invention may be delivered with other therapeutics for treating depression. For instance, the subject may be co-administered an Anti-Depressant with the compounds of the invention. Co-administered refers to an administration that may be delivered at the same time in the same vehicle, at the same time in a different vehicle or at a different time, either before or after the compounds of the invention. Examples of anti-depressants and guidance on their use for treating depression is found, for example, in Baldessarini, R. J., (2006), Drug Therapy of Depression and Anxiety, Chapter 17 in Goodman & Gilman's, The Pharmacological Basis of Therapeutics, eleventh edition, Brunton, L. L., et al, (ed.), Magraw-Hill, New York, pages 429-459, the content of which is incorporated herein by reference in its entirety. Preferred anti-depressants for combination treatment are dopamine, agonists thereof, serotonin enhancing agents, e.g., SSRIs, serotonin, 5-hydroxytryptophan, and tryptophan, tricyclics, e.g., phenothiazines, tertiary amine tricyclics and secondary amine tricyclics, and atypical anti-depressants. Examples of SSRIs include, without limitation, fluoxetine and paroxetine. Examples of tertiary amine tricyclics include, without limitation, amitriptylen, doxepin, imipramine and trimipramine. Examples of secondary amine tricyclics include, without limitation, amoxapine, desipramine and nortiptyline. Examples of atypical anti-depressants (including atypical anti-psychotics) include, without limitation, duloxetine, and mirtazapine.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

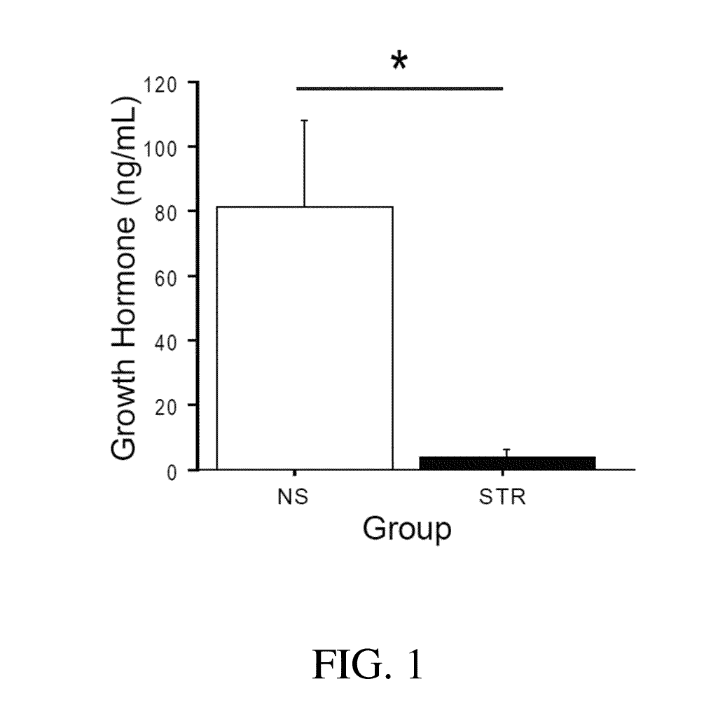

It is not known how GH is affected by chronic stress. To address this, GH levels in the dorsal hippocampus of rats were examined after either 14 consecutive days of immobilization stress (STR) or daily handling (no stress, or NS). Hippocampal GH was dramatically downregulated following chronic stress (FIG. 1; group: F(1,6)=8.29, p<0.05). This shows that acute and chronic stress can produce very different effects on the brain.

### Example 1

### Examining the Association Between Loss of Growth Hormone in the Hippocampus and Stress-Related Changes in Hippocampal Function

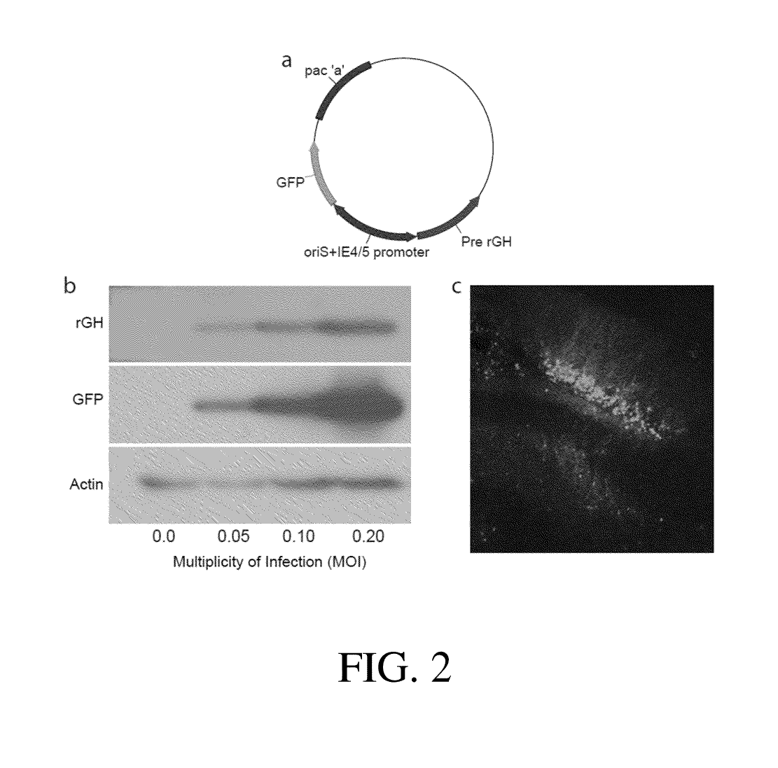

In order to determine whether the loss of GH in the hippocampus was associated with stress-related changes in hippocampal function, an HSV-1 based amplicon was first constructed, in which the full-length gene for rat presomatotropin (rGH), the precursor molecule for GH (31), was co-expressed with green florescent protein (GFP) under the control of a viral promoter (FIG. 2A). This amplicon, as well as a control amplicon expressing only GFP, was packaged into replication-defective HSV viral vectors. These vectors were then used to infect dorsal hippocampus (FIG. 2C). A representative infection, showing high levels of expression in pyramidal cells of CAL is shown.

Methods:

Amplicon Construction:

The rat presomatotropin gene was cloned as an 818 bp HindIII cut fragment from the p-RGH1 plasmid (31), provided by Dr. Douglas Weigent (University of Alabama at Birmingham), into the HindIII cloning site of the HSV amplicon plasmid pα22GFP (32), in which a bicistronic HSV-based promoter simultaneously drives expression of a transgene from the α-4 promoter and enhanced green florescent protein (eGFP) from the α-22 promoter. The pα22GFP plasmid was used as a control.

Virus Preparation

Virus was generated using standard methods (33). Briefly, plasmids were amplified to generate endotoxin-free DNA, which was transfected into 2-2 cells. The next day, cells were superinfected with 5dl1.2 helper virus. After two days, the cells were sonicated and centrifuged to release infectious viral particles. The resulting supernatant was twice passaged onto 2-2 cells. After the final sonication and centrifugation, the supernatant was purified on a sucrose gradient, pelleted, and resuspended in 10% sucrose in D-PBS. Aliquots of each amplicon were stored at −80° C. till use. Amplicon titers were ˜1×10⁸ IU/ml.

Subjects:

All experiments used adult male Long-Evans rats (225-275 g, Taconic, Germantown, N.Y.), housed individually (68-72° F.; 12-h light-dark cycle, 7 AM lights on). Rodent chow and water was provided ad libitum. Stressed and unstressed animals were housed in separate cubicles. All procedures were in accordance with the US National Institutes of Health (NIH) Guide for the Care and Use of Laboratory Animals and were approved by the MIT Institutional Animal Care and Use Committee, and the Animal Care and Use Review Office of the Army Research Office.

Stereotactic Virus Delivery:

Surgery was performed 18-24 h following the final handling or immobilization stress session. Rats were anesthetized (with either Nembutal at 65 mg/kg, or a ketamine:xylazine:acepromazine cocktail at 100:100:10 mg/kg, i.p.) and mounted in a stereotaxic frame. Small holes were drilled for intra-cranial placement of the injector aimed within the dorsal hippocampus: A/P−3.3, M/L+/−2.0, D/V−3.2, relative to brain surface and bregma(34). Virus was infused with either pulled glass pipettes or 33 g stainless steel bevel needles attached to a 10 ul Hamilton syringe (Hamilton Company, Reno, Nev.). The pipettes or syringes were mounted in stereotaxic barrel holder, and the rate of virus delivery was controlled by a syringe pump (Harvard Apparatus, Holliston, Mass.). Virus was infused at 0.1 ul/m for 20 m (2 ul total volume per hemisphere). Injectors remained in the brain for 10 m before being withdrawn. Incisions were closed with wound clips and Ketoprofen (1 mg, s.c.) was administered for pain and inflammation.

Protein (Western) Immunoblot:

Vero cells were plated in 6 cm dishes using standard methods (33). Purified virus was used to infect cells at multiplicities of infection ranging from 0 to 0.2. After three days, cells were harvested and homogenized. Protein was loaded on to gels for electrophoretic transfer. Membranes were incubated, in succession, with the following primary antibodies overnight at 4° C.: 1:5000 rabbit anti-GH (National Hormone and Peptide Program, NIDDK), 1:500 mouse anti-GFP (Roche, Indianapolis, Ind.), 1:1000 mouse anti-Actin (Millipore; Billerica, Mass.). Following incubation in secondary antibody, immunoreactivity was visualized using chemiluminescent detection.

Growth Hormone ELISA:

Hippocampi were homogenized 1:6 in homogenization buffer (2% HALT protease cocktail and 0.15% NP-40 in PBS) using a LabGEN 125 homogenizer (Cole-Parmer; Vernon Hills, Ill.) for 8-10 s on ice. After 5 m of incubation on ice, tubes were spun at 18,000 g for 20 m at 4° C. and the supernatant was transferred to a new tube. The resulting solution was assayed as per manufacturer's protocol (Millipore; Billerica, Mass.).

Results:

Using an antibody against rGH, confirmed that GH was expressed by cultured cells infected with the GH viral vector (FIG. 2B).

### Example 2

### The Role of GH in Chronic Stress-Related Changes in Auditory Trace Fear Conditioning

The role of GH in chronic stress-related changes was first examined in auditory trace fear conditioning, a hippocampus-dependent task (36). Rats were repeatedly exposed to daily immobilization stress (STR) or handling (NS). One day later, rats received intra-hippocampal infusions of either GH or GFP virus. After three days for recovery, rats were subjected to auditory trace fear conditioning. Over the following two days, long-term contextual fear memory and auditory trace fear memory were assessed.

Methods:

Subjects:

Rats were treated as described in Example 2.

Experimental Methods:

Growth hormone ELISA, Protein (Western) Immunoblot methods are as described in Example 2.

Immobilization Stress:

Immobilization stress was administered for 4 h per day for 10 (contextual fear conditioning experiment) or 14 (trace fear conditioning experiment) consecutive days Animals were placed in Decapicone plastic bags (Braintree Scientific; Braintree, Mass.), which were secured at the tail to keep the bagged animal in an upright position. Stress occurred in an isolated lab room, separate from all behavioral testing space. All stress sessions were performed between 10 AM and 4 PM. Unstressed control rats were handled daily for 30 s.

Pavlovian Fear Conditioning:

All behavioral testing commenced 72 h after stereotactic viral delivery, a time point corresponding to maximal transgene expression with HSV-based viral vectors (33). Fear conditioning experiments were conducted in a modified chamber (MED Associates; St. Albans, Vt.) housed in a sound-attenuating cubicle. For auditory trace fear conditioning, rats were placed in individual chambers in a novel context (house and room lights on, 1% acetic acid, grid floors) for five minutes before receiving tone (20 s, 2 kHz, 85 dB)-footshock (1 s, 0.85 mA) pairings, with the stimuli separated by a 35 s trace interval. A 3 m inter-trial interval was used. Long-term contextual fear memory was assessed twenty-four hours later, when the rats were returned to the chambers for a 5 m context extinction test. Long-term auditory fear memory was measured twenty-four hours later; the rats were placed in the chamber configured as a novel context (room and house lights off, 0.3% Pine Sol odor, white Plexiglas floor and wall inserts). Animals were allowed 3 m to habituate to the chamber before four tones (85 dB, 2 kHz) were presented with inter-trial intervals of 3 m35 s. For contextual fear conditioning, animals were placed in a novel context (house and room lights on, 1% acetic acid, grid floors) for three minutes before receiving three unsignaled footshocks (2 s, 0.5 mA) separated by a 90 s inter-stimulus interval. Long-term contextual memory was measured 24 h later, when the rats were returned to the context for an 8 m context test. Infrared video was recorded throughout all sessions. Freezing was measured offline using commercial software (VideoFreeze, MedAssociates, St. Albans, Vt.).

Histology:

Following completion of the experiment, animals were anesthetized with an overdose of isoflurane and the brains were removed from the cranium. Brains were bisected along the midline. The dorsal hippocampus was dissected from one hemisphere, placed in a sterile eppendorf tube, and flash frozen in dimethylbutane on dry ice. The tissue was stored at −80° C. The other hemisphere was placed in 4% paraformaldehyde for 72 h then transferred to a 30% sucrose/4% paraformaldehyde solution for a minimum of 3 days. Fixed tissue was cut into coronal sections on a cryostat (40 μm) and mounted on slides. Sections were assessed for GFP florescence. Animals with incorrect placements were excluded from all analyses.

Results:

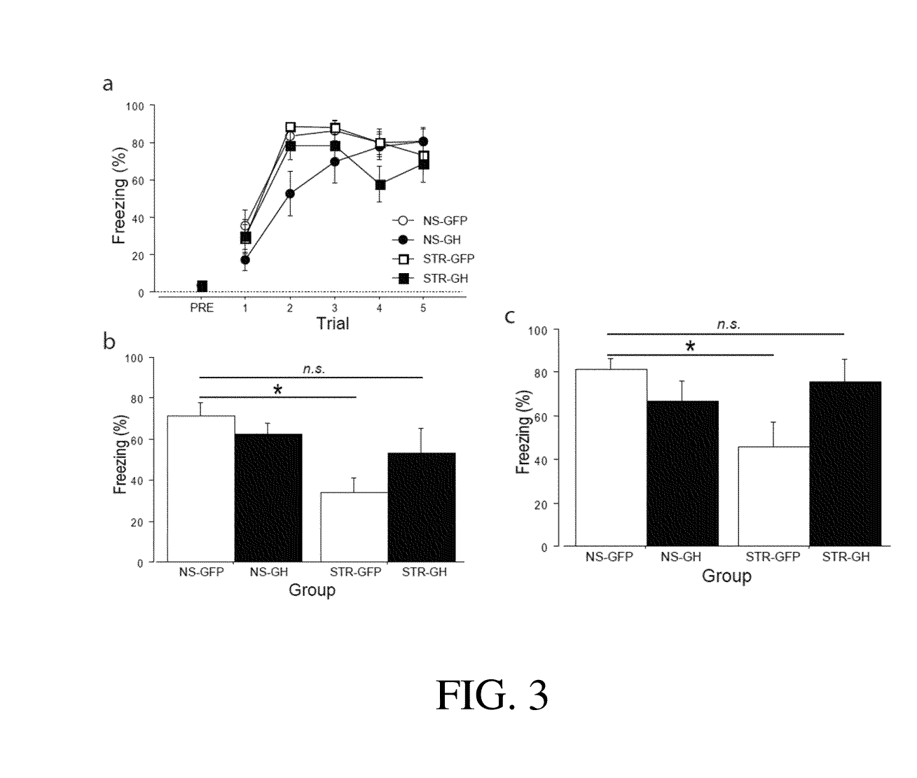

Stress did not affect the rapid acquisition of auditory trace fear conditioning (FIG. 3A; stress: F(1,25)=0.02, p=ns), and this was not differentially impacted by GH expression (Infusion×Stress interaction: F(1,25)=0.17, p=ns). In contrast, the effects of GH expression on long-term contextual and trace auditory fear memory did depend on stress (FIG. 3B; Infusion×Stress interaction: F(1,25)=3.22, p=0.08; and FIG. 3C; Infusion×Stress interaction: F(1,25)=5.99, p<0.05). Whereas rats in the STR-GFP group showed lower levels of conditional freezing than rats in the NS-GFP group, rats in the STR-GH group displayed levels of conditional freezing that were statistically indistinguishable from those displayed by rats in the NS-GFP group (FIG. 3B-C, post-hoc comparisons). These results show that the impairments in hippocampal function following chronic stress can be attributed to the loss of hippocampal GH.

### Example 3

### The Role of GH in Stress-Related Changes in Foreground Contextual Fear Conditioning

To further investigate this, the role of GH in stress-related changes was also examined in foreground contextual fear conditioning, a hippocampus-dependent task (37). Rats were repeatedly exposed to daily immobilization stress (STR) or handling (NS). One day later, rats received intra-hippocampal infusions of either GH or GFP virus. After recovering for three days, rats were subjected to contextual fear conditioning. Long-term contextual memory was measured the next day.

Methods:

Subjects:

Rats were treated as described in Example 2.

Experimental Methods:

Growth hormone ELISA, Protein (Western) Immunoblot and Histology methods are as described in Example 2 Immobilization stress and Pavlovian Fear Conditioning were administered as described in the Methods of Example 2.

Results:

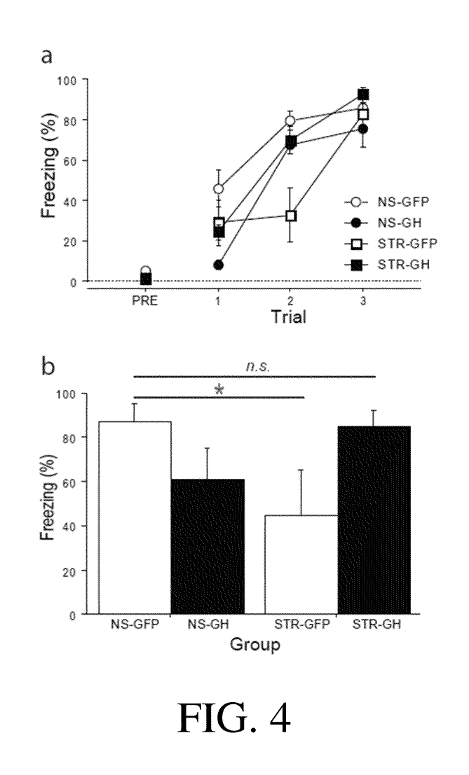

The effects of stress on contextual fear acquisition were dependent on the type of virus that had been infused in the hippocampus (FIG. 4A; Stress×Infusion interaction, F(1,10)=1.35, p<0.01): rats in the STR-GFP group displayed slower acquisition than rats in the STR-GH group (post-hoc comparisons). In contrast, rats in the NS-GFP and NS-GH groups acquired fear at virtually identical rates (post-hoc comparisons). Similar effects of GH were observed for long-term contextual memory (FIG. 4B; Stress×Infusion interaction, F(1,10)=5.29, p<0.05): intra-hippocampal GH rescued the memory-impairing effects of stress, leading to conditional freezing levels indistinguishable from NS-GFP controls (post-hoc comparisons). However, intra-hippocampal GH in unstressed controls produced a mild impairment in conditional freezing, relative to NS-GFP controls (p=0.09; post-hoc comparison). These results further confirm that a loss in hippocampal GH contributes to stress-related impairment in hippocampal function.

The examples provided herein demonstrate that chronic stress induces a profound and lasting downregulation of GH in the dorsal hippocampus. Animals that experienced chronic stress also exhibited significant impairment on two hippocampus-dependent tasks. When GH levels were increased in stressed animals using viral-mediated gene transfer, the animals did not exhibit any stress-related decrements in performance. This shows that a stress-induced loss of hippocampal GH contributes to stress-related impairment in hippocampal function, and that restoration of GH after stress termination is sufficient to reverse these changes.

Because GH can potentiate hippocampal synaptic plasticity, the overexpression of GH may lead to enhancement of hippocampal function. Interestingly, overexpression of GH in the hippocampus of unstressed animals had minimal effect on contextual or trace fear conditioning. For animals subjected to contextual fear conditioning, there was a mild trend for unstressed animals to have an impairment in long-term contextual fear memory when GH was overexpressed in hippocampus. This may be due to an occlusion effect, where GH may transiently saturate plasticity in the hippocampus such that synapses may not be further potentiated by learning. However, overexpression of GH clearly did not produce a broad occlusion of further hippocampus-dependent learning. The lack of occlusion may result from GH regulating its own expression, and viral expression of recombinant GH may have downregulated expression of endogenous GH. Regardless, these results support GH as a novel target for pharmacological intervention following stress in diseases such as depression, and show that interventions that boost GH signaling in hippocampus after stress may promote stress resilience.

### Example 4

### Amygdalar Growth Hormone is Increased by Chronic Stress

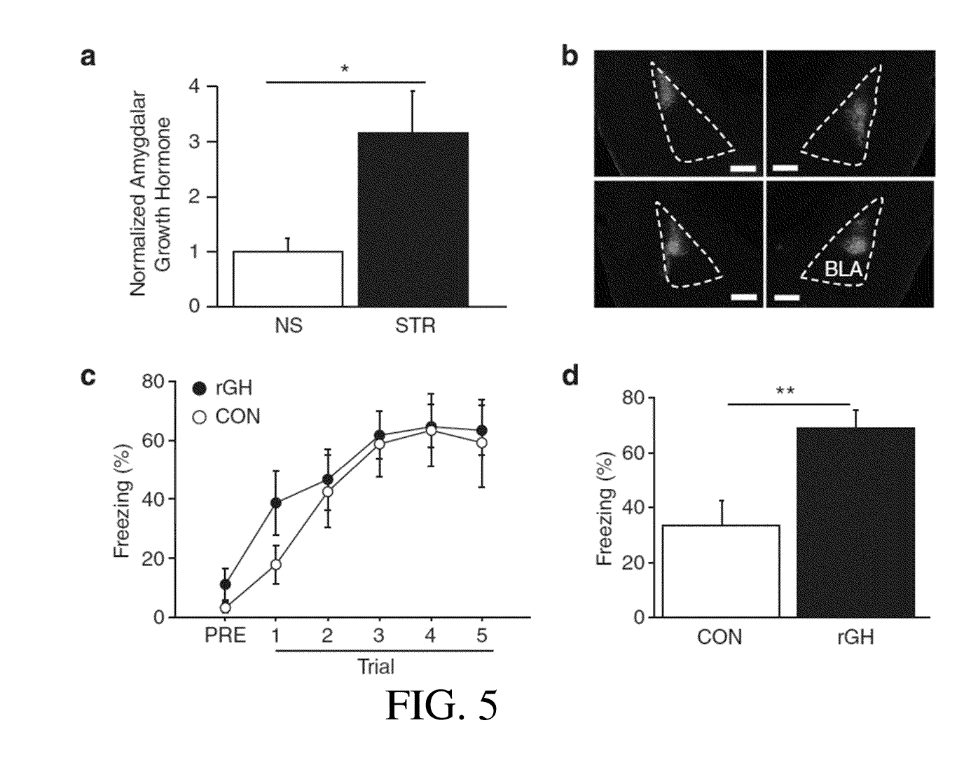

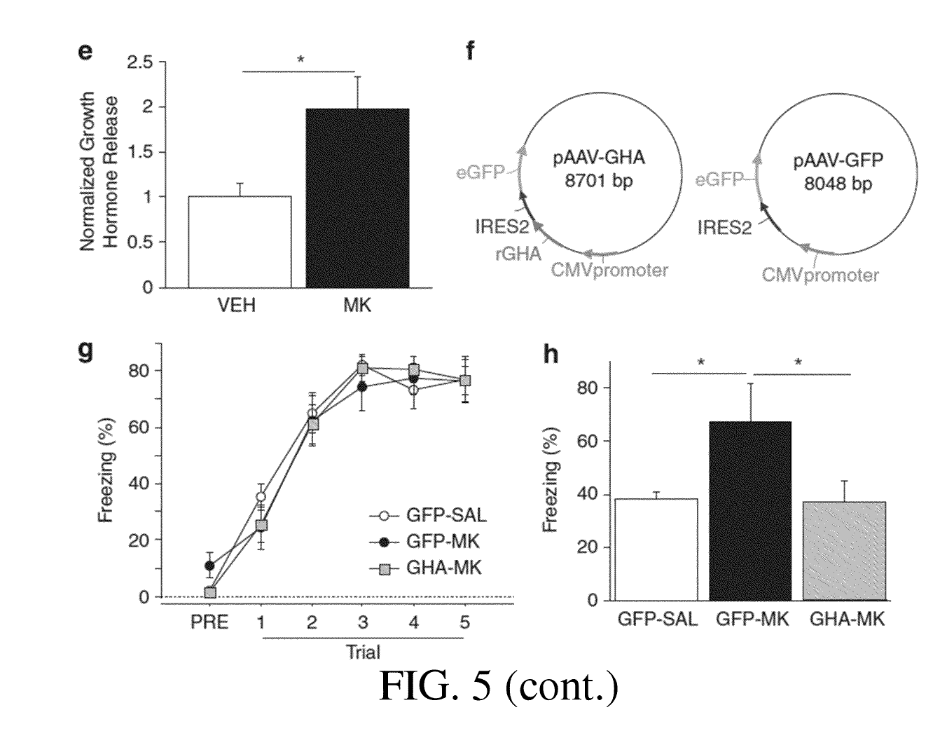

Growth hormone, a major effector of the ghrelin receptor, interacts with ghrelin in the amygdala to enhance fear memory. Although the pituitary expresses the highest levels of GH, it is also expressed in other brain regions, including the BLA.⁴⁴ It is not known how prolonged stress alters GH in the BLA. To test this, the impact of repeated immobilization stress (STR) or daily handling (NS) on GH levels in the BLA were examined. GH was readily detected in BLA homogenate and significantly upregulated 24 h after chronic stress (FIG. 5a, group: F(1, 16)=6.44, P<0.05), the time point at which increases are observed in circulating ghrelin and fear conditioning. This suggests that GH-mediated signaling in the BLA may be amplified following stress.

GH can induce synaptic plasticity⁴⁶ and is increased in response to learning,⁴⁷ but it is unclear how it affects amygdala function. Herpes simplex-based viral vectors were used to express recombinant rat GH and a GFP reporter or GFP only.⁵¹ Naive rats received intra-BLA infusions of either the recombinant rat GH virus or the GFP-only control virus (CON) (FIG. 5b). After 3 days, when herpes simplex virus-mediated transgene expression is at its maximum,⁴⁸ auditory fear conditioning was administered. Fear to the tone was assessed 48 h later. Overexpression of recombinant rat GH did not alter fear acquisition (FIG. 5c; infusion×trial interaction: F(4, 52)=0.57, P=ns) but did enhance long-term fear memory (FIG. 5d, infusion: F(1, 13)=9.97, P<0.01). These data demonstrate that high levels of GH in the BLA are sufficient to enhance fear learning, an effect that is similar to the effect of repeated intra-BLA ghrelin receptor stimulation.

To determine whether ghrelin receptor stimulation in amygdala triggers the release of GH as it does from the pituitary,⁴³ short-term cultures were generated of BLA cells and measured GH protein in the media following treatment with either MK-0677 or vehicle. Stimulation of the ghrelin receptor in BLA cells led to significantly elevated release of GH (FIG. 5e; treatment: F(1, 8)=8.24, P<0.05). These results show that ghrelin receptor stimulation can trigger GH release from the amygdala.

Because ghrelin and GH can interact in amygdala, we next determined whether GH is a necessary downstream signaling partner for the fear-enhancing effects of repeated ghrelin receptor stimulation. An AAV viral construct to overexpress a mutant form of the rat GH protein (GHA) that acts as a functional antagonist to endogenous GH was generated.^{49,50} Following infusion of AAV to overexpress either GHA or a control protein (GFP) (FIG. 5f), rats were permitted to recover for 5 weeks. After recovery, rats that were infused with GHA received daily injections of either the ghrelin receptor agonist MK-0677 (MK) or saline (SAL) for 10 days. Rats that were infused with GFP received daily injections of SAL for 10 days. At 24 h after the final injection, all rats were subjected to auditory fear conditioning, followed by assessment of long-term auditory fear memory in a subsequent session 48 h later. Although no differences were observed between any group during fear conditioning (FIG. 5g; group: F(2, 13)=0.07; to P=ns), differences in long-term fear memory were observed (FIG. 5h; group: F(2, 13)=4.32, P<0.05). Specifically, antagonizing the activity of GH prevented the fear-enhancing effects of repeated ghrelin receptor agonism (FIG. 5h; planned comparisons between GFP-SAL vs GFP-MK and GFP-MK vs GHA-MK). These data reveal that repeated ghrelin receptor stimulation requires GH-dependent signaling in the amygdala to exert its fear-enhancing effects.

### Example 5

### Hippocampal Growth Hormone Promotes Increased Spine Density

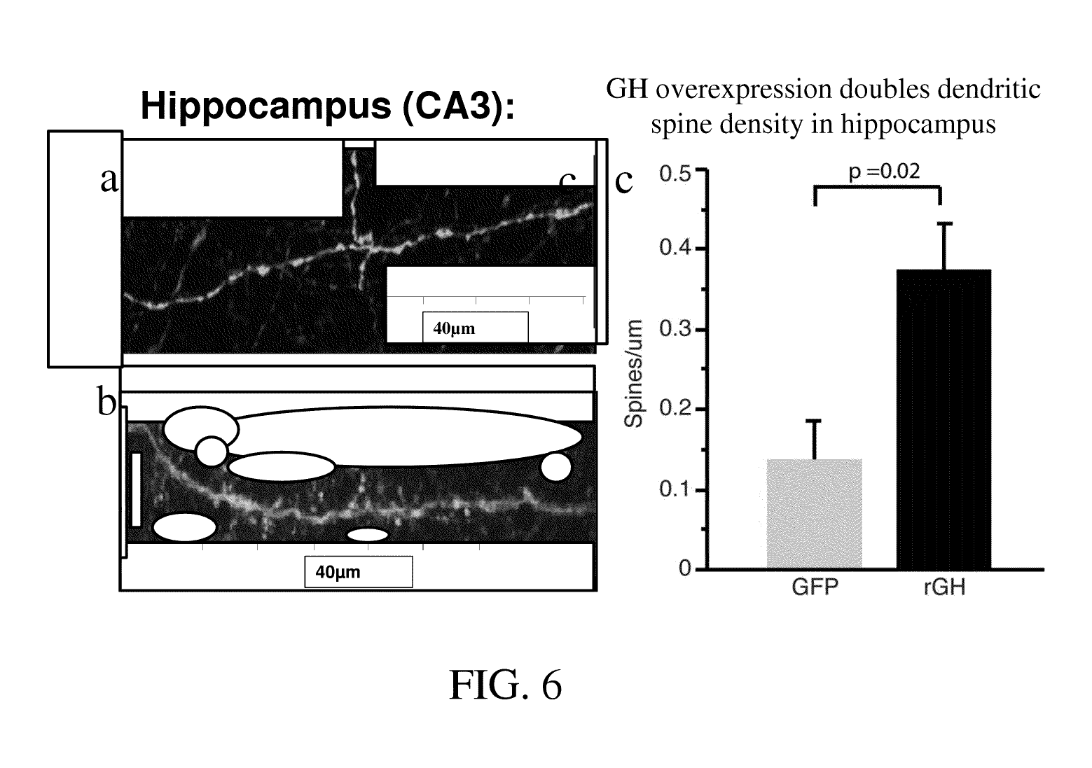

We have demonstrated that increasing GH levels in the hippocampus leads to increased spine density. Because stress and depression both cause hippocampal atrophy, this suggests that hippocampal GH counteracts stress and depression. The data is shown in FIG. 6. FIGS. 6a and 6b show images of increased spine density in the hippocampus. A graph depicting the results of GH overexpression on dendritic spine density is shown in FIG. 6c. GH overexpression doubles dendritic spine density in hippocampus.

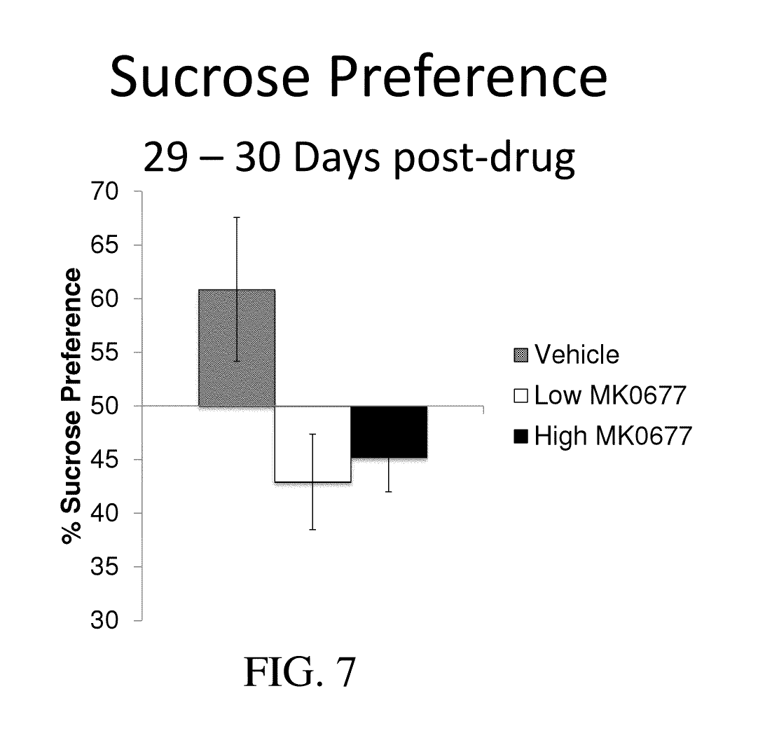

Sucrose preference (measuring hedonia) as a measure of depression with low dose MK0677 or high dose MK0677 was also examined. Low and high doses of the ghrelin receptor agonist, MK0677, led to anhedonia. Repeated ghrelin receptor stimulation (mimicking one effect of chronic stress, which is to enhance the circulating levels of ghrelin and thus ghrelin signaling) induces depressive behaviors in rats. The results are shown in FIG. 7.

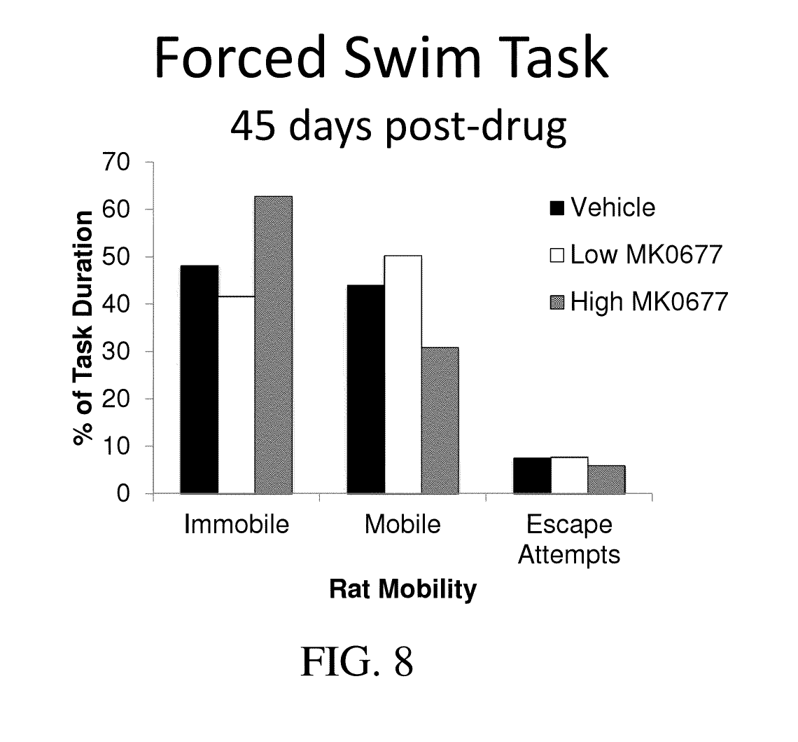

Forced swim as a measure of depression was also examined. A high dose of the ghrelin receptor agonist led to immobility in the forced swim, which is seen as an analog of “giving up” in depressed humans. The results are shown in FIG. 8. Enhancing hippocampal function has been shown to counteract these behaviors in rodents. Repeated ghrelin receptor stimulation (mimicking one effect of chronic stress, which is to enhance the circulating levels of ghrelin and thus ghrelin signaling) induces depressive behaviors in rats.

### REFERENCES

1. B. McEwen and J. C. Wingfield: Allostasis and allostatic load. In: Encyclopedia of Stress. Ed G. Fink. Academic Press, New York (2007)
2. R. P. Juster, G. Bizik, M. Picard, G. Arsenault-Lapierre, S. Sindi, L. Trepanier, M. F. Marin, N. Wan, Z. Sekerovic, C. Lord, A. J. Fiocco, P. Plusquellec, B. S. McEwen and S. J. Lupien: A transdisciplinary perspective of chronic stress in relation to psychopathology throughout life span development. Dev Psychopathol, 23(3), 725-76 (2011) doi:S0954579411000289 [pii] 10.1017/S0954579411000289
3. B. S. McEwen: Protective and damaging effects of stress mediators. N Engl J Med, 338(3), 171-9 (1998) doi:10.1056/NEJM199801153380307
4. C. Mazure: Does Stress Cause Psychiatric Illness? American Psychiatric Press, Inc., Washington, D.C. (1995)
5. F. Lederbogen, P. Kirsch, L. Haddad, F. Streit, H. Tost, P. Schuch, S. Wust, J. C. Pruessner, M. Rietschel, M. Deuschle and A. Meyer-Lindenberg: City living and urban upbringing affect neural social stress processing in humans. Nature, 474(7352), 498-501 (2011) doi:nature10190 [pii] 10.1038/nature10190
6. J. K. Belanoff, M. Kalehzan, B. Sund, S. K. Fleming Ficek and A. F. Schatzberg: Cortisol activity and cognitive changes in psychotic major depression. Am J Psychiatry, 158(10), 1612-6 (2001)
7. A. Jeneson and L. R. Squire: Working memory, long-term memory, and medial temporal lobe function. Learn Mem, 19(1), 15-25 (2012) doi:19/1/15 [pii] 10.1101/lm.024018.111
8. D. A. Bangasser and T. J. Shors: The hippocampus is necessary for enhancements and impairments of learning following stress. Nat Neurosci, 10(11), 1401-3 (2007)
9. K. A. Goosens: Hippocampal regulation of aversive memories. Curr Opin Neurobiol, 21(3), 460-6 (2011) doi:50959-4388(11)00048-1 [pii] 10.1016/j.conb.2011.04.003
10. T. J. Shors, C. Chua and J. Falduto: Sex differences and opposite effects of stress on dendritic spine density in the male versus female hippocampus. J Neurosci, 21(16), 6292-7 (2001)
11. Y. Chen, C. M. Dube, C. J. Rice and T. Z. Baram: Rapid loss of dendritic spines after stress involves derangement of spine dynamics by corticotropin-releasing hormone. J Neurosci, 28(11), 2903-11 (2008) doi:28/11/2903 [pii] 10.1523/JNEUROSCI.0225-08.2008
12. T. J. Shors: Acute stress rapidly and persistently enhances memory formation in the male rat. Neurobiol Learn Mem, 75(1), 10-29 (2001) doi:10.1006/nlme 1999.395651074-7427(99)93956-4 [pii]
13. D. J. de Quervain, B. Roozendaal and J. L. McGaugh: Stress and glucocorticoids impair retrieval of long-term spatial memory. Nature, 394(6695), 787-90 (1998) doi: 10.1038/29542
14. D. M. Diamond, A. M. Campbell, C. R. Park, J. Halonen and P. R. Zoladz: The temporal dynamics model of emotional memory processing: a synthesis on the neurobiological basis of stress-induced amnesia, flashbulb and traumatic memories, and the Yerkes-Dodson law. Neural Plast, 60803 (2007)
15. A. M. Magarinos and B. S. McEwen: Stress-induced atrophy of apical dendrites of hippocampal CA3c neurons: involvement of glucocorticoid secretion and excitatory amino acid receptors. Neuroscience, 69(1), 89-98 (1995)
16. Y. Watanabe, E. Gould and B. S. McEwen: Stress induces atrophy of apical dendrites of hippocampal CA3 pyramidal neurons. Brain Res, 588(2), 341-5 (1992)
17. C. Sandi, H. A. Davies, M. I. Cordero, J. J. Rodriguez, V. I. Popov and M. G. Stewart: Rapid reversal of stress induced loss of synapses in CA3 of rat hippocampus following water maze training. Eur J Neurosci, 17(11), 2447-56 (2003)
18. A. Vyas, R. Mitra, B. S. Shankaranarayana Rao and S. Chattarji: Chronic stress induces contrasting patterns of dendritic remodeling in hippocampal and amygdaloid neurons. J Neurosci, 22(15), 6810-8 (2002)
19. R. Pawlak, B. S. Rao, J. P. Melchor, S. Chattarji, B. McEwen and S. Strickland: Tissue plasminogen activator and plasminogen mediate stress-induced decline of neuronal and cognitive functions in the mouse hippocampus. Proc Natl Acad Sci USA, 102(50), 18201-6 (2005)
20. J. Nishimura, Y. Endo and F. Kimura: A long-term stress exposure impairs maze learning performance in rats. Neurosci Lett, 273(2), 125-8 (1999)
21. J. K. Kleen, M. T. Sitomer, P. R. Killeen and C. D. Conrad: Chronic stress impairs spatial memory and motivation for reward without disrupting motor ability and motivation to explore. Behav Neurosci, 120(4), 842-51 (2006)
22. H. Lakshminarasimhan and S. Chattarji: Stress leads to contrasting effects on the levels of brain derived neurotrophic factor in the hippocampus and amygdala. PLoS One, 7(1), e30481 (2012) doi:10.1371/journal.pone.0030481PONE-D-11-18672 [pii]
23. F. Nyberg and P. Burman: Growth hormone and its receptors in the central nervous system—location and functional significance. Horm Res, 45(1-2), 18-22 (1996)
24. L. Y. Sun, K. Al-Regaiey, M. M. Masternak, J. Wang and A. Bartke: Local expression of GH and IGF-1 in the hippocampus of GH-deficient long-lived mice. Neurobiol Aging,
26(6),929-37(2005)doi: S01974580(04)002842[pii] 10.1016/j.neurobiolaging.2004.07.010 25. N. R. Zearfoss, J. M. Alarcon, P. Trifilieff, E. Kandel and J. D. Richter: A molecular circuit composed of CPEB-1 and c-Jun controls growth hormone-mediated synaptic plasticity in the mouse hippocampus. J Neurosci, 28(34), 8502-9 (2008) doi:28/34/8502 [pii] 10.1523/JNEUROSCI.1756-08.2008
26. G. S. Mahmoud and L. M. Grover: Growth hormone enhances excitatory synaptic transmission in area CA1 of rat hippocampus. J Neurophysiol, 95(5), 2962-74 (2006) doi:00947.2005 [pii] 10.1152/jn.00947.2005
27. D. P. Molina, O. J. Ariwodola, C. Linville, W. E. Sonntag, J. L. Weiner, J. K. Brunso-Bechtold and M. M. Adams: Growth hormone modulates hippocampal excitatory synaptic transmission and plasticity in old rats. Neurobiol Aging, 33(9), 1938-49 (2012) doi:S0197-4580(11)00351-4 [pii] 10.1016/j.neurobiolaging.2011.09.014
28. C. P. Donahue, R. V. Jensen, T. Ochiishi, I. Eisenstein, M. Zhao, T. Shors and K. S. Kosik: Transcriptional profiling reveals regulated genes in the hippocampus during memory formation. Hippocampus, 12(6), 821-33 (2002)
29. M. N. Treacy, F. Ryan and F. Martin: Functional glucocorticoid inducible enhancer activity in the 5′-flanking sequences of the rat growth hormone gene. J Steroid Biochem Mol Biol, 38(1), 1-15 (1991) doi:0960-0760(91)90395-L [pii]
30. C. P. Donahue, K. S. Kosik and T. J. Shors: Growth hormone is produced within the hippocampus where it responds to age, sex, and stress. Proc Natl Acad Sci USA, 103(15), 6031-6 (2006)
31. P. H. Seeburg, J. Shine, J. A. Martial, J. D. Baxter and H. M. Goodman: Nucleotide sequence and amplification in bacteria of structural gene for rat growth hormone. Nature, 270(5637), 486-94 (1977)
32. D. Kaufer, W. O. Ogle, Z. S. Pincus, K. L. Clark, A. C. Nicholas, K. M. Dinkel, T. C. Dumas, D. Ferguson, A. L. Lee, M. A. Winters and R. M. Sapolsky: Restructuring the neuronal stress response with anti-glucocorticoid gene delivery. Nat Neurosci, 7(9), 947-53 (2004)
33. P. Lim and R. Neve: Generation of High-Titer Defective HSV-1 Vectors. In: Current Protocols in Neuroscience. John Wiley and Sons, Inc., (2000)
34. G. Paxinos and C. Watson: The Rat Brain in Stereotaxic Coordinates—The New Coronal Set, Fifth Edition. Elsevier Academic Press, San Diego (2005)
35. T. J. Shors, C. Weiss and R. F. Thompson: Stress-induced facilitation of classical conditioning. Science, 257(5069), 537-9 (1992)
36. J. D. Raybuck and K. M. Lattal: Double dissociation of amygdala and hippocampal contributions to trace and delay fear conditioning. PLoS One, 6(1), e15982 doi:10.1371/journal.pone.0015982
37. S. G. Anagnostaras, G. D. Gale and M. S. Fanselow: Hippocampus and contextual fear conditioning: recent controversies and advances. Hippocampus, 11(1), 8-17 (2001) doi: 10.1002/1098-1063 (2001)11:1<8::AID-HIPO1015>3.0.CO; 2-7
38. M. Le Greves, P. Steensland, P. Le Greves and F. Nyberg: Growth hormone induces age-dependent alteration in the expression of hippocampal growth hormone receptor and N-methyl-D-aspartate receptor subunits gene transcripts in male rats. Proc Natl Acad Sci USA, 99(10), 7119-23 (2002) doi:10.1073/pnas.09213539999/10/7119 [pii]
39. Y. P. Tang, E. Shimizu, G. R. Dube, C. Rampon, G. A. Kerchner, M. Zhuo, G. Liu and J. Z. Tsien: Genetic enhancement of learning and memory in mice. Nature, 401(6748), 63-9 (1999) doi:10.1038/43432
40. M. I. Ransome and A. M. Turnley: Growth hormone signaling and hippocampal neurogenesis: insights from genetic models. Hippocampus, 18(10), 1034-50 (2008) doi: 10.1002/hipoo.20463
41. B. Vollmayr, C. Simonis, S. Weber, P. Gass and F. Henn: Reduced cell proliferation in the dentate gyrus is not correlated with the development of learned helplessness. Biol Psychiatry, 54(10), 1035-40 (2003) doi:S0006322303005274 [pii]
42. M. Fleshner, S. F. Maier, D. M. Lyons and M. A. Raskind: The neurobiology of the stress-resistant brain. Stress, 14(5), 498-502 (2011) doi:10.3109/10253890.2011.596865
43. Kojima M, Hosoda H, Date Y, Nakazato M, Matsuo H, Kangawa K. Ghrelin is a growth-hormone-releasing acylated peptide from stomach. Nature 1999; 402:656-660.
44. Donahue C P, Kosik K S, Shors T J. Growth hormone is produced within the hippocampus where it responds to age, sex, and stress. Proc Natl Acad Sci USA 2006; 103: 6031-6036.
45. Pacold S T, Kirsteins L, Hojvat S, Lawrence A M. Biologically active pituitary hormones in the rat brain amygdaloid nucleus. Science 1978; 199: 804-806.
46. Mahmoud G S, Grover LM. Growth hormone enhances excitatory synaptic transmission in area CA1 of rat hippocampus. J Neurophysiol 2006; 95: 2962-2974.
47. Donahue C P, Jensen R V, Ochiishi T, Eisenstein I, Zhao M, Shors T et al. Transcriptional profiling reveals regulated genes in the hippocampus during memory formation. Hippocampus 2002; 12: 821-833.
48. Carlezon Jr. W A, Thome J, Olson V G, Lane-Ladd S B, Brodkin E S, Hiroi N et al. Regulation of cocaine reward by CREB. Science 1998; 282: 2272-2275.
49. Chen W Y, Wight D C, Mehta B V, Wagner T E, Kopchick J J. Glycine 119 of bovine growth hormone is critical for growth-promoting activity. Mol Endocrinol 1991; 5: 1845-1852.
50. Chen W Y, Wight D C, Wagner T E, Kopchick J J. Expression of a mutated bovine growth hormone gene suppresses growth of transgenic mice. Proc Natl Acad Sci USA 1990; 87: 5061-5065.
51. Vander Weele C M, Saenz C, Yao J, Correia S S, Goosens K A. Restoration of hippocampal growth hormone reverses stress-induced hippocampal impairment. Front Behav Neurosci 2013; 7: 66.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

All references, patents and patent applications disclosed herein are incorporated by reference with respect to the subject matter for which each is cited, which in some cases may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for treating depression in a subject, comprising administering to a subject having depression a growth hormone or an agonist thereof in an effective amount to treat depression.

2. The method of claim 1 wherein the subject is administered growth hormone.

3. The method of claim 1, wherein the subject is administered a growth hormone agonist.

4. The method of claim 1, wherein the growth hormone agonist is a nucleic acid vector, wherein the nucleic acid vector encodes growth hormone or functional analogs thereof.

5. The method of claim 1, wherein the growth hormone or agonist thereof is delivered to the hippocampus.

6. The method of claim 1, wherein the depression is a depressive disorder, a major depressive disorder, complicated grief, dysthymia, post-partum depression, or psychotic depression.

7. The method of claim 1, wherein the depression is associated with chronic stress.

8. The method of claim 1, wherein the growth hormone or agonist thereof is administered systemically.

9. The method of claim 7, wherein the growth hormone or agonist thereof is administered while the subject is experiencing the stress.

10. The method of claim 7, wherein the growth hormone or agonist thereof is administered only during the time that the subject is experiencing the stress.

11. The method of claim 1, wherein the growth hormone or agonist thereof is administered to the subject in a sustained release device.

12. The method of claim 1, wherein the growth hormone or agonist thereof is administered to the subject orally.

13. The method of claim 1, wherein the growth hormone or agonist thereof is administered to the subject intravenously.

14. The method of claim 7, wherein the growth hormone or agonist thereof is administered before, during and/or after exposure of the subject to chronic stress.
